SEQUENCE LISTING (2 sequences)

SEQ ID NO: 1 - DNA, 19 bp, K. pneumoniae
  Carbapenemase
atgtcactgtatcgccgtc

SEQ ID NO: 2 - DNA, 21 bp, K. pneumoniae
  Carbapenemase
ttactgcccgttgacgcccaa

(19) United States
(12) Patent Application Publication
(10) Pub. No.: US 2014/0128364 A1
(43) Pub. Date: May 8, 2014

(54) THERAPEUTIC USES OF TIGEMONAM AND CARUMONAM

(71) Applicant: REMPEX PHARMACEUTICALS, San Diego, CA (US)

(72) Inventor: Olga Rodny, Mill Valley, CA (US)

(73) Assignee: Rempex Pharmaceuticals, San Diego, CA (US)

(21) Appl. No.: 13/797,509

(22) Filed: Mar. 12, 2013

Related U.S. Application Data
(60) Provisional application No. 61/722,685, filed on Nov. 5, 2012.

(51) Int. Cl. A61K 31/427

(52) U.S. Cl. CPC A61K 31/427
USPC 514/210.15; 435/252.1; 435/252.8; 435/253.3

(57) ABSTRACT

Disclosed herein is the use of tigemonam and carumonam in treating bacterial infection caused by bacteria producing K. pneumoniae carbapenemase (KPC) enzymes.

### INCORPORATION BY REFERENCE TO ANY PRIORITY APPLICATIONS

This application claims the benefit of priority to U.S. Provisional Patent Application Ser. No. 61/722,685, filed Nov. 5, 2012, which is herein incorporated by reference in its entirety. Any and all applications for which a foreign or domestic priority claim is identified in the Application Data Sheet as filed with the present application, or any correction thereto, are hereby incorporated by reference under 37 CFR 1.57.

### SEQUENCE LISTING

The present application is being filed along with a Sequence Listing in electronic format. The Sequence Listing is provided as a file entitled REMPEX_{—}097A.TXT, created Mar. 12, 2013 which is 1 kilobyte in size. The information in the electronic format of the Sequence Listing is incorporated herein by reference in its entirety.

### BACKGROUND OF THE INVENTION

1. Field of the Invention

The present invention relates to the field of chemistry and medicine. More particularly, the present invention relates to the use of the monobactams tigemonam and carumonam to treat bacterial infections.

2. Description of the Related Art

Antibiotics have been effective tools in the treatment of infectious diseases during the last half-century. From the development of antibiotic therapy to the late 1980s there was almost complete control over bacterial infections in developed countries. However, in response to the pressure of antibiotic usage, multiple resistance mechanisms have become widespread and are threatening the clinical utility of anti-bacterial therapy. The increase in antibiotic resistant strains has been particularly common in major hospitals and care centers. The consequences of the increase in resistant strains include higher morbidity and mortality, longer patient hospitalization, and an increase in treatment costs.

Various bacteria have evolved β-lactam deactivating enzymes, namely β-lactamases, that counter the efficacy of the various β-lactam antibiotics. The β-lactamases can be grouped into four classes based on their amino acid sequences—classes A, B, C, and D. These enzymes catalyze the chemical degradation of β-lactam antibiotics, rendering them inactive. Some β-lactamases can be transferred within and between various bacterial strains and species. The rapid spread of bacterial resistance and the evolution of multi-resistant strains severely limit β-lactam treatment options available.

New β-lactamases have recently evolved that hydrolyze the carbapenem class of antimicrobials, including imipenem, biapenem, doripenem, meropenem, and ertapenem, as well as other β-lactam antibiotics. The class A K. pneumoniae carbapenemases (KPC) is a group of recently identified carbapenemases and has been reported in the U.S., France, Greece, U.K. and many other countries. Biochemical data showed that KPC enzymes hydrolyze many β-lactam antibiotics including penicillins, cephalosporins, and aztreonam. (Nordmann P. et al., Lancet Infect Dis 2009, 9(4):228-36; Queenan A M & Bush K., Clin Microbial Rev 2007, 20(3): 440, 58). Treatment of bacterial strains resistant to these carbanpenems can be associated with poor outcome. Thus, there is a need for antibiotics resistant to the KPC-type β-lactamases.

### SUMMARY OF THE INVENTION

The present invention relates to the use of monobactams Tigemonam and Carumonam to treat bacterial infections.

Some embodiments relate to a method of treating infection in a subject caused by a bacteria expressing K. pneumoniae carbapenemases (KPC), the method comprising administering to a subject in need thereof an effective amount of tigemonam.

Some embodiments include a method of treating infection in a subject caused by a bacterial expressing K. pneumoniae carbapenemases (KPC) comprising administering to a subject in need thereof an effective amount of carumonam.

Some embodiments include a method of inhibiting bacterial growth, the method comprising contacting a bacteria expressing K. pneumoniae carbapenemases (KPC) with an effective amount of tigemonam.

Some embodiments include a method of inhibiting bacterial growth, the method comprising contacting a bacteria expressing K. pneumoniae carbapenemases (KPC) with an effective amount of carumonam.

In some embodiments, the KPC is KPC-2 or KPC-3.

Some embodiments include a method of treating a bacterial infection, the method comprising administering aztreonam to a subject in need thereof, determining that the infection is resistant to treatment with aztreonam, and subsequently administering an effective amount of tigemonam to the subject.

Some embodiments include a method of treating a bacterial infection that is resistant to aztreonam, the method comprising administering to a subject in need thereof an effective amount of tigemonam.

Some embodiments include a method of treating a bacterial infection, the method comprising administering aztreonam to a subject in need thereof, determining that the infection is resistant to treatment with aztreonam, and subsequently administering an effective amount of carumonam to the subject.

Some embodiments relate to a method of treating a bacterial infection that is resistant to aztreonam, the method comprising administering to a subject in need thereof an effective amount of carumonam.

In some embodiments, the bacteria is selected from Citrobacter freundii, Escherichia coli, Enterobacter cloacae, or Klebsiella pneumonia.

In some embodiments, the bacteria is selected from Pseudomonas aeruginosa, Pseudomonas fluorescens, Stenotrophomonas maltophilia, Salmonella typhimurium, Salmonella typhi, Salmonella paratyphi, Salmonella enteritidis, Shigella dysenteriae, Shigella flexneri, Shigella sonnei, Enterobacter aerogenes, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.

### DETAILED DESCRIPTION OF THE PREFERRED EMBODIMENT

Some embodiments relate to tigemonam and carumonam and their therapeutic use in treating bacterial infections caused by a bacteria expressing K. pneumoniae carbapenemases (KPC). The KPC enzymes are a group of recently identified carbapenemases that belong to molecular class A β-lactamases. There are ten known variants of KPC-type β-lactamases, KPC-2 through KPC-11, which differ from one another by one or two amino acid substitutions. (Tapp, R.& Urban, C.; J. Human Pharmacology and Drug Therapy, 2012, 32, 5, 399, which is incorporated herein by reference in its entirety). In common with other class A β-lactamases, these highly proficient KPC enzymes have an efficient hydrolysis mechanisms involving a catalytic residue, which deactivates β-lactam substrates.

The KPC enzymes are capable of hydrolyzing penicillins, cephalosporins and aztreonam. (Yigit H, Antimicrob Agents Chemother. 2001; 45(4):1151-1161). The KPC enzymes can also confer resistance to many carbapenems, including meropenem, imipenem, ertapenem and doripenem. Their worldwide spread makes them a potential threat to currently available antibiotic-based treatments.

Tigemonam and carumonam are monobactams belonging to the same antibiotic group as the commercially available aztreonam.

Some embodiments include methods of treating a bacterial infection in a subject caused by a bacteria expressing a KPC enzyme by administering to the subject an effective amount of tigemonam or carumonam. In various embodiments, the bacteria expresses one or more of KPC-2, KPC-3, KPC-4, KPC-5, KPC-6, KPC-7, KPC-8, KPC-9, KPC-10, or KPC-11. In some embodiments, the bacteria expresses KPC-2. In some embodiments, the bacteria expresses KPC-3.

Some embodiments include a method of treating a bacterial infection, the method comprising administering aztreonam to a subject in need thereof, determining that the infection is resistant to treatment with aztreonam, and subsequently administering an effective amount of tigemonam or carumonam to the subject. For example, in some embodiments, a physician prescribes a course of aztreonam to treat a bacterial infection. After or during the course of administration, the physician determines that the subject demonstrates no significant improvement in the symptoms of infection or less improvement than expected. The physician then subsequently administers an effective amount of tigemonam or carumonam.

Some embodiments include methods of treating a bacterial infection that is resistant to aztreonam by administering to a subject an effective amount of tigemonam or carumonam.

The bacteria expressing the KPC enzyme can be from a variety of species and strains. For example, although KPC enzymes evolved in Klebsiella pneumonia, they can and have migrated to other species. Thus, in some embodiments, the KPC-expressing bacteria is selected from the group consisting of Citrobacter freundii, Escherichia coli, Enterobacter cloacae, Klebsiella pneumonia, Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtherias, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus. In one embodiment, the bacteria is Citrobacter freundii. In one embodiment, the bacteria is Escherichia coli. In one embodiment, the bacteria is Enterobacter cloacae. In one embodiment, the bacteria is Klebsiella pneumonia.

In some embodiments, the subject is a human.

Further embodiments include administering a combination of medicaments to a subject in need thereof. A combination can include a combination of tigemonam and carumonam. A combination can also include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered intravenously.

### DEFINITIONS

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

The term “resistance” or “resistant” as used herein refers to a bacterial strain displaying delayed, lessened and/or null response to an antibiotic agent. For example, after treatment with aztreonam, the bacterial load of a subject infected with a aztreonam-resistant bacteria may be reduced to a lesser degree compared to the amount in bacterial load reduction exhibited by a subject infected with a non-resistant strain.

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

### Administration and Pharmaceutical Composition

The compounds tigemonam and carumonam are administered at a therapeutically effective dosage. While human dosage levels have yet to be optimized, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of tigemonam or carumonam can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

Tigemonam or carumonam can be formulated into pharmaceutical compositions for use in treatment of the conditions described herein. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of tigemonam or carumonam; and (b) a pharmaceutically acceptable carrier, diluent, excipient or combination thereof.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The following examples will further describe the present invention, and are used for the purpose of illustration only, and should not be considered as limiting.

### EXAMPLES

### Example 1

### Microbiological Activities of Aztreonam, Tigemonam, and Carumonam Against Engineered Bacterial Strains

Isogenic Escherichia coli strains carrying plasmids that contain genes encoding either KPC-2 or KPC-3 β-lactamases were used to compare the microbiological activities of aztreonam, tigemonam and carumonam. Minimum inhibitory concentration (MIC) values were determined using Clinical and Laboratory Standards Institute (CLSI) broth microdilution methodology as described in CLSI document M07-A9 (2012). Aztreonam, tigemonam, and carumonam (concentration range, 0.125-128 μg/ml) were tested in a 96-well format using cation-adjusted Mueller-Hinton broth. MICs were recorded after an eighteen-hour growth at 35° C. Table 1 lists the MICs of aztreonam, tigemonam, and carumonam for Escherichia coli strains expressing KPC-type β-lactamases. In Table 1, all genes were cloned into vector pUCP24 and introduced into Escherichia coli strain. The data demonstrate that KPC-2 and KPC-3 confer less resistance to tigemonam and carumonam as compared to aztreonam when produced in engineered Escherichia coli strain.

TABLE 1
MICs of monobactams for Escherichia coli strains expressing KPC-type
β-lactamases

| Strain | Organism | β-Lactamase | Aztreonam | Tigemonam | Carumonam |
|---|---|---|---|---|---|
| ECM6704 | Escherichia coli | Vector alone | <=0.125 | <=0.125 | <=0.125 |
| ECM6701 | Escherichia coli | KPC-2 | 16 | 0.25 | 0.25 |
| ECM6702 | Escherichia coli | KPC-3 | 32 | 0.5 | 0.25 |

### Example 2

### Microbiological Activities of Aztreonam, Tigemonam, and Carumonam Against Bacterial Strains of Clinical Origin

The presence of genes encoding KPC β-lactamase in each strain was confirmed using PCR. Table 2 lists the PCR primers for testing the presence of KPC genes. SEQ ID NO. 1 and SEQ ID NO. 2 are used to amplify a section of the KPC genes.

TABLE 2
PCR Primers for testing KPC gene presence

| SEQ | | |
|---|---|---|
| ID | | |
| NO | | Sequence (from 5′ to 3′) |
| 1 | Forward primer | atgtcactgtatcgccgtc |
| 2 | Reverse primer | ttactgcccgttgacgcccaa |

To prepare the template, a fresh single colony was incubated overnight at 37° C. on Luria Bertani (LB) agar and resuspended in 100 μl of sterile water. 0.5 μl of this culture was used as a template for 10 μl PCR reaction. The PCR reaction mixture consisted of 5.0 μl DreamTaq Green PCR Master Mix (2×) (Fermantas) or AmpliTaq Gold 360 Master Mix (2×) (Life Technology), 0.5 μl primer pair (each at 10 uM), 0.5 μl template, and 4.0 μl water. The thermo-cycling conditions were 5 minutes at 94° C. for the first cycle, followed by 35 cycles of 94° C. for 30 seconds, 55° C. for 30 seconds, and 68° C. for 1.5 minutes, and 7 minutes at 68° C. for the last cycle.

The PCR reaction mixture was run on agarose gel (1%). The KPC gene was confirmed by the presence of a PCR product that is the same as the positive control. After the presence of KPC was confirmed, MICs for aztreonam, tigemonam, and carumonam were determined using Clinical and Laboratory Standards Institute (CLSI) broth microdilution methodology as described in CLSI document M07-A9 (2012). Aztreonam, tigemonam and carumonam (concentration range, 0.125-128 μg/ml) were tested in a 96-well format using cation-adjusted Mueller-Hinton broth. MICs were recorded after an eighteen-hour growth at 35° C. Table 2 shows the MICs for aztreonam, tigemonam and carumonam against selected clinical strains expressing KPC-type β-lactamases. As shown in Table 3, tigemonam and carumonam have lower MICs than aztreonam and thus are more potent than aztreonam against clinical strains producing KPC-2 or KPC-3.

TABLE 3
MICs of Aztreonam, Tigemonam and Carumonam for selected clinical strains
expressing KPC-type β-lactamases

| Strain | Organism | β-Lactamase | Aztreonam | Tigemonam | Carumonam |
|---|---|---|---|---|---|
| CF1012 | Citrobacter | KPC-2 | >64 | 8 | 2 |
| | freundii | | | | |
| ECL1026 | Enterobacter | KPC-2, | 64 | 4 | 2 |
| | cloacae | TEM-1 | | | |
| ECL1036 | Enterobacter | KPC-3, | >64 | 8 | 2 |
| | Vcloacae | TEM-1 | | | |
| ECL1055 | Enterobacter | KPC-3, | >64 | 8 | 4 |
| | loacae | TEM | | | |
| EC1007 | Escherichia coli | KPC-3 | >64 | 1 | 1 |
| KP1008 | Klebsiella | KPC-2 | 64 | 1 | 0.5 |
| | pneumoniae | | | | |

### Example 3

### Kinetics of Aztreonam, Tigemonam and Carumonam Hydrolysis

Purified KPC-2 enzymes were used to determine the kinetic parameters of azteronam, tigemonam and carumonam hydrolysis. For carumonam and tigemonam, 377 nM purified KPC-2 was mixed with corresponding monobactam in 50 mM sodium phosphate (pH 7.0) and 0.1 mg/ml bovine serum albumin (reaction buffer). 24 nM purified KPC-2 was mixed with aztreonam in 50 mM sodium phosphate (pH 7.0) and 0.1 mg/ml bovine serum albumin (reaction buffer). The concentrations of each monobactam varied from 1600 μM to 3.65 μM with 1.5× increments between dilutions. The optical density of the mixture was monitored at 318 nm and at 37° C. immediately after the enzyme was mixed with the corresponding monobactam. The optical density was checked every 2 minutes for 2 hours. Initial rates of monobactam degradation were calculated using the following extinction coefficients: 420 M-1×cm-1 for carumonam, 416 M-1×cm-1 for tigemonam, 660 M-1×cm-1 for aztreonam. Vmax and Km values were calculated using “Prizm” software (“GraphPad”). Kcat values were calculated using the equation kcat=Vmax/[enzyme concentration]. Table 4 shows the hydrolysis rate of aztreonam, carumonam, and tigemonam by purified KPC-2 β-lactamases. As demonstrated in Table 4, KPC-2 is about 100-fold more efficient in hydrolyzing aztreonam than hydrolyzing tigemonam and carumonam.

TABLE 4
Hydrolysis rate of Aztreonam, Carumonam, and Tigemonam by KPC-2 β-
lactamases

| Kinetic parameters | Aztreonam | Carumonam | Tigemonam |
|---|---|---|---|
| Kₘ, μM | 700.9 | 602.7 | 678.0 |
| Vₘₐₓ, μM/min | 64.9 | 20.3 | 5.4 |
| k_{cat}, min⁻¹ | 2759.2 | 54 | 14 |
| k_{cat}/Kₘ, μM⁻¹ × min⁻¹ | 3.9 | 0.09 | 0.02 |

Although the invention has been described with reference to embodiments and examples, it should be understood that numerous and various modifications can be made without departing from the spirit of the invention. Accordingly, the invention is limited only by the following claims.

What is claimed is:

1. A method of treating infection in a subject caused by a bacteria expressing a K. pneumoniae carbapenemase (KPC), comprising administering to a subject in need thereof an effective amount of tigemonam.

2. The method of claim 1, wherein the KPC is KPC-2 or KPC-3.

3. The method of claim 1, wherein the bacteria is selected from Citrobacter freundii, Escherichia coli, Enterobacter cloacae, or Klebsiella pneumonia.

4. The method of claim 1, wherein the bacteria is selected from Pseudomonas aeruginosa, Pseudomonas fluorescens, Stenotrophomonas maltophilia, Salmonella typhimurium, Salmonella typhi, Salmonella paratyphi, Salmonella enteritidis, Shigella dysenteriae, Shigella flexneri, Shigella sonnei, Enterobacter aerogenes, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.

5. A method of treating infection in a subject caused by a bacteria expressing a K. pneumoniae carbapenemase (KPC), comprising administering to a subject in need thereof an effective amount of carumonam.

6. The method of claim 5, wherein the KPC is KPC-2 or KPC-3.

7. The method of claim 5, wherein the bacteria is selected from Citrobacter freundii, Escherichia coli, Enterobacter cloacae, or Klebsiella pneumonia.

8. The method of claim 5, wherein the bacteria is selected from Pseudomonas aeruginosa, Pseudomonas fluorescens, Stenotrophomonas maltophilia, Salmonella typhimurium, Salmonella typhi, Salmonella paratyphi, Salmonella enteritidis, Shigella dysenteriae, Shigella flexneri, Shigella sonnei, Enterobacter aerogenes, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.

9. A method of inhibiting bacterial growth, comprising contacting a bacteria expressing a K. pneumoniae carbapenemase (KPC) with an effective amount of tigemonam.

10. The method of claim 9, wherein the KPC is KPC-2 or KPC-3.

11. The method of claim 9, wherein the bacteria is selected from Citrobacter freundii, Escherichia coli, Enterobacter cloacae, or Klebsiella pneumonia.

12. The method of claim 9, wherein the bacteria is selected from Pseudomonas aeruginosa, Pseudomonas fluorescens, Stenotrophomonas maltophilia, Salmonella typhimurium, Salmonella typhi, Salmonella paratyphi, Salmonella enteritidis, Shigella dysenteriae, Shigella flexneri, Shigella sonnei, Enterobacter aerogenes, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.

13. A method of inhibiting bacterial growth, comprising contacting a bacteria expressing a K. pneumoniae carbapenemase (KPC) with an effective amount of carumonam.

14. The method of claim 13, wherein the KPC is KPC-2 or KPC-3.

15. The method of claim 13, wherein the bacteria is selected from Citrobacter freundii, Escherichia coli, Enterobacter cloacae, or Klebsiella pneumonia.

16. The method of claim 13, wherein the bacteria is selected from Pseudomonas aeruginosa, Pseudomonas fluorescens, Stenotrophomonas maltophilia, Salmonella typhimurium, Salmonella typhi, Salmonella paratyphi, Salmonella enteritidis, Shigella dysenteriae, Shigella flexneri, Shigella sonnei, Enterobacter aerogenes, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.

17. A method of treating a bacterial infection, comprising:
administering aztreonam to a subject in need thereof;
determining that the infection is resistant to treatment with aztreonam; and
subsequently administering an effective amount of tigemonam to the subject.

18. The method of claim 17, wherein the bacteria is selected from Citrobacter freundii, Escherichia coli, Enterobacter cloacae, or Klebsiella pneumonia.

19. The method of claim 17, wherein the bacteria is selected from Pseudomonas aeruginosa, Pseudomonas fluorescens, Stenotrophomonas maltophilia, Salmonella typhimurium, Salmonella typhi, Salmonella paratyphi, Salmonella enteritidis, Shigella dysenteriae, Shigella flexneri, Shigella sonnei, Enterobacter aerogenes, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.

20. A method of treating a bacterial infection that is resistant to aztreonam, comprising administering to a subject in need thereof an effective amount of tigemonam.

21. The method of claim 20, wherein the bacteria is selected from Citrobacter freundii, Escherichia coli, Enterobacter cloacae, or Klebsiella pneumonia.

22. The method of claim 20, wherein the bacteria is selected from Pseudomonas aeruginosa, Pseudomonas fluorescens, Stenotrophomonas maltophilia, Salmonella typhimurium, Salmonella typhi, Salmonella paratyphi, Salmonella enteritidis, Shigella dysenteriae, Shigella flexneri, Shigella sonnei, Enterobacter aerogenes, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.

23. A method of treating a bacterial infection, comprising:
administering aztreonam to a subject in need thereof;
determining that the infection is resistant to treatment with aztreonam; and
subsequently administering an effective amount of carumonam to the subject.

24. The method of claim 23, wherein the bacteria is selected from Citrobacter freundii, Escherichia coli, Enterobacter cloacae, or Klebsiella pneumonia.

25. The method of claim 23, wherein the bacteria is selected from Pseudomonas aeruginosa, Pseudomonas fluorescens, Stenotrophomonas maltophilia, Salmonella typhimurium, Salmonella typhi, Salmonella paratyphi, Salmonella enteritidis, Shigella dysenteriae, Shigella flexneri, Shigella sonnei, Enterobacter aerogenes, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.

26. A method of treating a bacterial infection that is resistant to aztreonam, comprising administering to a subject in need thereof an effective amount of carumonam.

27. The method of claim 26, wherein the bacteria is selected from Citrobacter freundii, Escherichia coli, Enterobacter cloacae, or Klebsiella pneumonia.

28. The method of claim 26, wherein the bacteria is selected from Pseudomonas aeruginosa, Pseudomonas fluorescens, Stenotrophomonas maltophilia, Salmonella typhimurium, Salmonella typhi, Salmonella paratyphi, Salmonella enteritidis, Shigella dysenteriae, Shigella flexneri, Shigella sonnei, Enterobacter aerogenes, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.
